(19) United States
(12) Patent Application Publication
(10) Pub. No.: US 2007/0113298 A1
(43) Pub. Date: May 17, 2007

(54) Genetically determined mouse model of resistance to transplantable cancers

(76) Inventors: Zheng Cui, Winston Salem, NC (US); Mark C. Willingham, Summerville, SC (US)

Correspondence Address: MYERS BIGEL SIBLEY & SAJOVEC, PO BOX 37428, RALEIGH, NC, 27627, US

(21) Appl. No.: 11/586,227

(22) Filed: Oct. 25, 2006

Related U.S. Application Data
(63) Continuation of application No. 10/827,654, filed on Apr. 19, 2004.

(51) Int. Cl. A01K 67/027; C12N 5/06

(52) U.S. Cl. 800/18; 435/354

(57) ABSTRACT

We have established and studied a colony of mice with a unique trait of host resistance to both ascites and solid cancers induced by transplantable cells. One dramatic manifestation of this trait is age-dependent spontaneous regression of advanced cancers. This powerful resistance segregates as a single-locus dominant trait, is independent of tumor burden and is effective against cell lines from multiple types of cancer. During spontaneous regression or immediately following exposure, cancer cells provoke a massive infiltration of host leukocytes which form aggregates and rosettes with tumor cells. The cytolytic destruction of cancer cells by innate leukocytes is rapid and specific without apparent damage to normal cells. The mice are healthy, cancer-free and have a normal life span. These observations suggest a previously unrecognized mechanism of immune surveillance that may have potential for therapy or prevention of cancer.

### RELATED APPLICATIONS

This application claims the benefit of U.S. provisional patent application Ser. No. 60/465,442, filed Apr. 24, 2003, the disclosure of which is incorporated by reference herein in its entirety.

This invention was made with Government support under grant number R55CA93868 from the National Cancer Institute and under grant number CA-09422 from the National Institute of Health. The Government has certain rights to this invention.

### FIELD OF THE INVENTION

The present invention concerns cancer resistant mice, mouse colonies, and methods of use thereof.

### BACKGROUND OF THE INVENTION

Regression of human cancers without treatment (spontaneous regression, SR) is well-documented for many types of cancer, but occurs infrequently (Bodey, B. et al., (1998). In Vivo 12, 107-122; Challis, G. B. & Stam, H. J. (1990). Acta Oncol. 29, 545-550; Cole, W. H. (1981). J. Surg. Oncol. 17, 201-209; Everson, T. C. (1967). Prog. Clin. Cancer 3, 79-95; Papac, R. J. (1998). In Vivo 12, 571-578). The most intriguing implication of SR is that there might be a rare, but extremely effective, mechanism engaged to eradicate cancer cells after the development of advanced malignancy. Despite efforts over many decades, the mechanism(s) of SR in humans and in animals has remained elusive.

Due to the absence of MHC, mouse S180 cells form highly aggressive cancers in all strains of laboratory mice (Alfaro, G. et al., (1992). Vet. Immunol. Immunopathol. 30, 385-398; Tarnowski, G. S. et al., (1973). Cancer Res. 33, 1885-1888), as well as in rats (Coffey, J. W. & Hansen, H. J. (1966). J. Immunol. 96, 1021-1026; Salaün, J. (1968). Eur. J. Cancer 4, 413-424). When injected into the peritoneal cavity, S180 cells grow exponentially with a generation time of 12-18 hours (Schiffer, L. M. et al. (1973). Cell Tissue Kinet. 6, 165-172). Growing primarily in suspension in the peritoneal cavity, S180 cells gradually plug peritoneal lymphatic drainage leading to accumulation of ascites fluid within 2 weeks. S180 cells can also metastasize into major organs near the peritoneal cavity, such as liver, kidney, pancreas, lung, stomach, and intestine (Cui and Willingham, unpublished data). Mice that develop ascites normally die in 3-4 weeks (10). S180-induced ascites represents one of the most aggressive transplantable cancers in experimental mouse models. Resistance to S180-induced ascites has never been reported. Due to their consistent response to transplanted S180 cells, BALB/c mice have become a standard strain for ascites production.

### SUMMARY OF THE INVENTION

In our laboratory, one male BALB/c mouse was unexpectedly found to remain ascites-free after repeated injections of S180 cells. We show here that this resistance (SR/CR) is germline transmissible and we describe the properties of this unique trait. A number of important applications arise from this finding, as discussed in greater detail below.

Accordingly, a first aspect of the present invention is a mouse that exhibits the phenotype of resistance to the development of ascites when tumor cells are injected into the peritoneal cavity of the mouse (e.g., when 2×10⁶ S180 tumor cells are injected into the peritoneal cavity of the mouse at 6 weeks of age). The mouse may be male or female. The mouse may exhibit, the phenotype of complete resistance to the development of ascites or the phenotype of spontaneous regression of ascites. In one embodiment, the mouse a BALB/c mouse.

A further aspect of the present invention is a mouse colony comprising a plurality of mice as described herein.

Mice of the present invention are useful for the production of cells as described herein, and for screening procedures as described herein.

Thus, a further aspect of the present invention is an isolated cell isolated from a mouse as described herein.

A still further aspect of the present invention is a method of producing a cancer-resistant mouse, comprising the steps of: (a) providing a first parent mouse and a second parent mouse, wherein the first parent mouse exhibits the phenotype of resistance to the development of ascites as described herein; and then (b) crossing the first and second parent mice with one another to produce a progeny mouse that exhibits a phenotype of resistance to the development of ascites as described herein.

The present invention is explained in greater detail in the drawings herein and the specification set forth below.

### BRIEF DESCRIPTION OF THE DRAWINGS

FIG. 1: A representative SR/CR BALB/c mouse (CR) was weighed daily for monitoring the presence of ascites after repeated injections (arrows) of S180 cells (2×10⁶ per injection) along with control BALB/c mice (WT) for each injection. All mice were 6 weeks old at the time of the first injection. The development of ascites in control mice was determined by a rapid increase of body weight and an enlarged abdomen.

FIG. 2: Pedigree analysis was performed by crossing S180-resistant BALB/c mice with S180-sensitive normal BALB/c mice. The progeny were weaned at 3 weeks and injected with 2×10⁶ S180 cells. Males are represented by squares and females by circles. Dark squares and circles are resistant mice. Clear circles represent control mice that are sensitive to S180 cells. Slashed squares and circles are S180-sensitive progeny.

FIG. 3: Survival analysis was performed by injecting either 5×10⁵ or 2×10⁶ S180 cells in the progeny from the cross of a CR BALB/c and an S180-sensitive C57BL/6. Fifteen litters consisting of a total of 107 mice were divided into two dosage groups. The injections were given at 6 weeks of age. The mice that developed ascites were marked as WT and ascites-free mice were marked as SR/CR. This demonstrates the lethality of S180 cells in the sensitive mice and uniform survival of the SR/CR mice. The number of mice in each group is shown in parentheses.

FIG. 4: The daily body weight graph representative of an S180-sensitive mouse (WT), a mouse with complete resistance to ascites (CR) and a mouse that underwent spontaneous regression of ascites (SR) is shown. Tumor regression in the SR phenotype occurred after day 14 and was complete at day 15.

FIG. 5: SR protected mice from developing ascites again upon repeated injection (second arrow) of S180 cells. Immediately after regression, one SR mouse and one control WT mouse were injected with 2×10⁶ S180 cells. The SR mouse failed to develop ascites again.

FIG. 6: The display of either the CR or the SR phenotypes is dependent on the age (in weeks) when the first injection of tumor cells was given. When the first injection was given at 6 weeks, 72 of 240 progeny (cross between SR/CR BALB/c and WT C57BL/6) show the CR phenotype. When the first injection was given at 12 weeks, 51 of 98 progeny were resistant, with 26 displaying the SR phenotype and 25 displaying the CR phenotype. When the first injection was given at 22 weeks, 31 of 120 progeny showed the SR phenotype and 5 displayed the CR phenotype. Four mice that had passed the resistant trait to their offspring developed ascites and died when the first injection of S180 cells was delayed until approximately 56 weeks.

FIG. 7: Shows the total number of cells of all types recovered from SR/CR or WT mice at different times after injection of S180 cells. Note the rapid influx of leukocytes at 6 hours in the SR mice, followed by a rapid decline.

FIG. 8: Total cells gradually increased in the WT mice, and these were mostly cancer cells as shown. In the SR/CR mice, no cancer cells remained after 3 days (arrow).

FIG. 9: Pedigree analysis of the CR and SR phenotypes.

### DETAILED DESCRIPTION OF THE PREFERRED EMBODIMENTS

Mice of the invention can be raised or propagated in accordance with known techniques, and cells, cell lines and tissue cultures obtained from such mice in accordance with known techniques, including but not limited to those described in U.S. Pat. No. 6,465,714, the disclosure of which is incorporated by reference herein in its entirety.

In general, mice of the present invention are created by (a) providing a first parent mouse and a second parent mouse, wherein the first parent mouse exhibits the phenotype of resistance to the development of ascites as described herein (e.g., when 2×10⁵ S180 tumor cells are injected into the peritoneal cavity of the mouse at 6 weeks of age); and then (b) crossing the first and second parent mice with one another to produce a progeny mouse that exhibits a phenotype of resistance to the development of ascites as described herein (e.g., when 2×10⁶ S180 tumor cells are injected into the peritoneal cavity of the mouse at 6 weeks of age). In one embodiment, the first parent mouse is a BALB/c mouse. In one embodiment, the second parent mouse is a BALB/c mouse. In one embodiment the first parent mouse is male and the second parent mouse is female; in another embodiment the first parent mouse is female and the second parent mouse is male. In one embodiment the second parent mouse is a wild-type mouse; in another embodiment the second parent mouse exhibits the phenotype of resistance to the development of ascites as described herein (e.g., when 2×10⁶ S180 tumor cells are injected into the peritoneal cavity of the mouse at 6 weeks of age). In still other embodiments the cancer resistant mouse is crossed to a cancer prone mouse to develop a mouse that is useful for studies of specific cancer development (e.g., prostate cancer, lung cancer, etc.).

As noted above, any of a variety of different cells can be harvested or collected from mice of the invention in accordance with known techniques to produce an isolated cell (or isolated cells) from a mouse as described herein (e.g., blood cells, hepatic cells, pancreatic cells, muscle cells, neural cells, skin cells, bone cells, hematopoietic stem cells, embryonic stem cells, egg cells, sperm cells, etc.). Cell cultures comprising, consisting of or consisting essentially of such cells may be propagated in accordance with known techniques, and tissue cultures comprising, consisting of or consisting essentially of such cells may be propagated or derived from such cells in accordance with known techniques. Such cells are useful for (1) providing mouse cells useful to screen in vitro for anti-cancer activity using human cancer samples (for example, to categorize human cancer types as to whether they are prone to this immune mechanism, something useful for clinical trials later); (2) for screening for human cancer types using in vivo tests using an cross of a mouse of the invention with a nude mouse; (3) for screening bacterial or other infectious agents for virulence in this type of immune system animal (mice of the invention appear to be more resistant to infectious agents in addition to cancer; (4) creating stem cells or hematopoietic cells for use by adoptive transfer to create the phenotype described herein in recipient mice (for other mouse strains) (creating a resistant mouse by cell transfer rather than by breeding)

Mice of the invention are useful for, among other things, chemical carcinogenesis screening, risk assessment in human populations for cancer, development of anti-microbial growth agents, development of anti-cancer and other therapies, particularly those involving immune regulation in aging animal development. Cells, cell lines and tissue cultures taken from or derived from mice of the invention are useful in like procedures, particularly in the screening potential carcinogenic agents, except embodied as in vitro rather than in vivo assays. Thus the present invention provides, among other things, a method of screening a compound for carcinogenic activity, comprising: (a) providing a mouse as described herein; (b) injecting cancer cells into the mouse as described herein (e.g., in an amount ordinarily insufficient to elicit the further growth of the cancer cells therein; or an amount sufficient to elicit further growth of the cancer cells therein but at a rate that permits comparative testing with control mice as described below); (c) administering the compound to the animal; and (d) determining whether the cancer cells grow in the animal by an amount greater than that seen in a corresponding control mouse that has been administered the same cancer cells but not been administered the compound, greater growth of the cancer cells indicating the compound may be carcinogenic.

A further aspect of the present invention is a method of screening a compound for anti-carcinogenic activity, comprising: (a) providing a mouse as described herein; (b) injecting cancer cells into the mouse as described herein (e.g., in an amount ordinarily sufficient to elicit the further growth of the cancer cells therein, or an amount sufficient to elicit further growth of the cancer cells therein but at a rate that permits comparative testing with control mice as described below); (c) administering the compound to the animal; and (d) determining whether the cancer cells grow in the animal by an amount less than that seen in a corresponding control mouse that has been administered the same cancer cells but not been administered the compound, less growth of the cancer cells indicating the compound may be anti-carcinogenic.

A further aspect of the present invention is a method of screening a compound for immune suppressing activity, comprising: (a) providing a mouse as described herein; (b) injecting cancer cells into the mouse as described herein (e.g., in an amount ordinarily insufficient to elicit the further growth of the cancer cells therein; or an amount sufficient to elicit further growth of the cancer cells therein but at a rate that permits comparative testing with control mice as described below); (c) administering said compound to said animal; and (d) determining whether said cancer cells grow in said animal by an amount greater than that seen in a corresponding control mouse that has been administered the same cancer cells but not been administered said compound, greater growth of the cancer cells indicating the compound may have immune suppressing activity.

A further aspect of the present invention is a method of screening a compound for immune stimulating activity, comprising: (a) providing a mouse as described herein; (b) injecting cancer cells into the mouse as described herein (e.g., in an amount ordinarily sufficient to elicit the further growth of the cancer cells therein, or an amount sufficient to elicit further growth of the cancer cells therein but at a rate that permits comparative testing with control mice as described below); (c) administering the compound to the animal; and (d) determining whether the cancer cells grow in the animal by an amount less than that seen in a corresponding control mouse that has been administered the same cancer cells but not been administered said compound, less growth of said cancer cells indicating said compound may have immune stimulating activity.

For purposes of the screening procedures described herein, any type of cancer cell may be injected or introduced into the animal into any suitable location of the animal, including but not limited to skin, lung, bone, colon, pancreas, prostate, or breast cancer cells of human, mouse, rat, monkey, or other animal origin; by any suitable route of administration including subcutaneous, intraperitoneal, or intrathecal injection, etc., in any suitable amount (e.g., from about 10, 1×10² or 1×10³ cells up to 1×10⁷; 1×10⁸; 1×10⁹; or 1×10¹⁰ cells or more, depending on the cells being injected, the route of administration, the purpose of the screening procedure, the age and condition of the subject, etc.).

The present invention is explained in greater detail in the following non-limiting Examples.

### EXAMPLE 1

Materials and Methods

Cell lines and mouse strains. Mouse cells were from American Type Culture Collection. Meth A sarcoma was a generous gift from Dr. Lloyd Old (Ludwig Institute for Cancer Research, New York Branch). Mouse cancer cells were propagated in culture according to the supplier's recommendations. BALB/c and C57BL/6 mice were from Charles River, and CAST/Ei and athymic C57BL/6^{foxn1/foxn1} nude mice were from The Jackson Lab. Mice were housed in plastic cages covered with air filter tops, containing hardwood shavings as bedding, allowed free access to water and regular chow and exposed to a 12 hr fluorescent light/dark cycle.

Cytoprep and Histology. Hematoxylin and DAPI staining of peritoneal cells washed from mice were standard procedures. For immunocytochemistry of surface markers, fixed cells in cytopreps were probed with anti-CD4, CD8, CD11c, or CD45, F4/80, Ly6G and NK1.1. These were followed by rhodamine-conjugated secondary antibodies and counted using a Zeiss Axioplan 2 fluorescence microscope.

Flow cytometry. Cells from peritoneal washes were stained with specific antibodies according to standard procedures recommended by the manufacturer and analyzed on a FACStar (BD Biosciences, Mountain View, Calif.). FSC and SSC gain settings were tuned to sort live cells from cell fragments.

Generation of S180 cells with GFP expression. The GFP-expressing vector was purchased from Invitrogen. S180 cells were transfected with the plasmid using the method of calcium phosphate precipitation and selected with 500 μg/ml G418. A strong GFP-expressing clone was selected using fluorescence microscopy and maintained with culture medium containing 500 μg/ml G418.

In Vitro Assay of Tumor Cell Lysis by Infiltrating Leukocytes. To induce peritoneal infiltration of tumor-killing leukocytes, the SR/CR mice were challenged with an i.p. injection of 2×10⁷ S180 cells 12 hour prior to peritoneal washes. Under anesthesia, the peritoneal cavity was infused with PBS or culture medium (DMEM) via an 18 G needle attached to a syringe. The wash solution was then thoroughly retrieved with the same needle and syringe resulting at least 95% of volume recovery. Since the wild type mice have only insignificant numbers of tumor-infiltrating leukocytes in response to the S180 challenge, splenocytes of wild type mice were isolated according to standard procedures as control effector cells; these showed no killing of S180 cells under the conditions used. To detect killing by leukocytes, we initially tested assays using ⁵¹Cr release, but SR/CR cell killing required >12 hr. in some cases, and the rate of leakage of ⁵¹Cr out of cells was too high to be useful. As an alternative, target cells were fluorescently labeled, prior to mixing with effector cells, by incubation with CellTracker Orange or DiO (Molecular Probes) at 39° C. for one hour in culture medium. Effector cells were mixed with target cells at a ratio of 50:1 and incubated for 24 hours at 39° C. to allow killing to occur. After incubation, the cell mixtures were also stained with Trypan blue to distinguish dead cells from live cells. Dead cells were positive for both Trypan blue staining and negative for fluorescence labeling (CellTracker or DiO). Live target cells were identified by their size, morphology and absence of Trypan blue staining. Target cell killing was interpreted as positive if over 50% of target cells were destroyed in 24 hours, when compared to a control consisting of target cells without effector cells. For generation of MethA tumors, 2×10⁶ cells were injected i.p. per mouse; these injections generated lethal ascites in both BALB/c and C57BL/6 control mice within 3 weeks.

Results

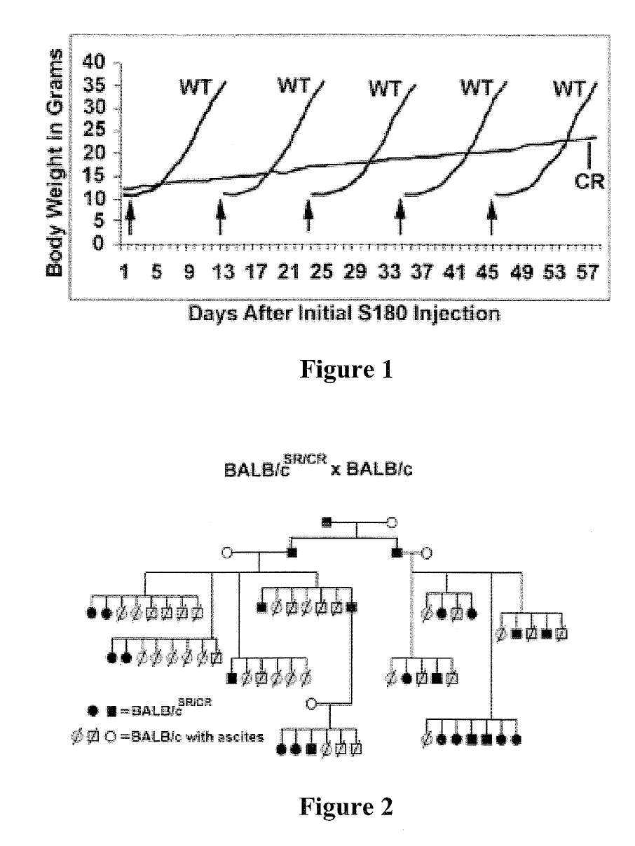

SR/CR Cancer Resistance Is Genetically Defined and Dose-Independent. An S180-resistant founder mouse was initially identified within a group of BALB/c mice as a result of its failure to develop ascites upon an injection of 5×10⁵ S180 cells. To verify that this failure was true resistance, the founder mouse was given two more injections of 2×10⁶ S180 cells, as were control BALB/c mice, followed by two further injections of 2×10⁷ S180 cells. No ascites developed in the founder mouse. This unique mouse remained healthy, cancer-free, and eventually died of old age at 26 months of age. This resistance was independent of tumor burden in the range tested (5×10⁵-2×10⁹ or up to 10% of total body weight,) and independent of whether the S180 cells had been passaged in vivo or through tissue culture. FIG. 1 shows typical changes of body weights in resistant and control mice that received similar injections of S180 cells. Additional injections of cells were given at ages 6, 12 and 18 months.

A breeding experiment was performed to determine if the cancer-resistant trait was germline transmissible in the BALB/c background (FIG. 2). Table I summarizes the results and genetic analysis from this breeding. The resistance phenotype was inherited in the F1 generation directly from crossing between resistant mice and S180-sensitive BALB/c mice, indicating that the phenotype was dominant to its wild type counterpart. The overall frequency of the SR/CR phenotype from outcrossing was ˜38%. This rate suggests strongly that only one locus is involved.

TABLE I

| Crosses | SR/CR | Total | % |
|---|---|---|---|
| BALB/c (WT) | 0 | >50 | 0 |
| C57BL/6 (WT) | 0 | >50 | 0 |
| BALB/c^{SR/CR} × BALB/c | 24 | 63 | 38 |
| BALB/c^{SR/CR} × C57BL/6 | 7 | 24 | 29 |
| (BALB/c^{SR/CR} × C57BL/6)^{SR/CR} × C57BL/6 | 43 | 122 | 35 |
| [(BALB/c^{SR/CR} × C57BL/6)^{SR/CR} × | 38 | 64 | 59 |
| C57BL/6]^{SR/CR} × C57BL/6 | | | |
| BALB/c^{SR/CR} × CAST/Ei | 13 | 39 | 33 |
| Total | 125 | 312 | 40 |
| Male | 54 | | |
| Female | 71 | | |

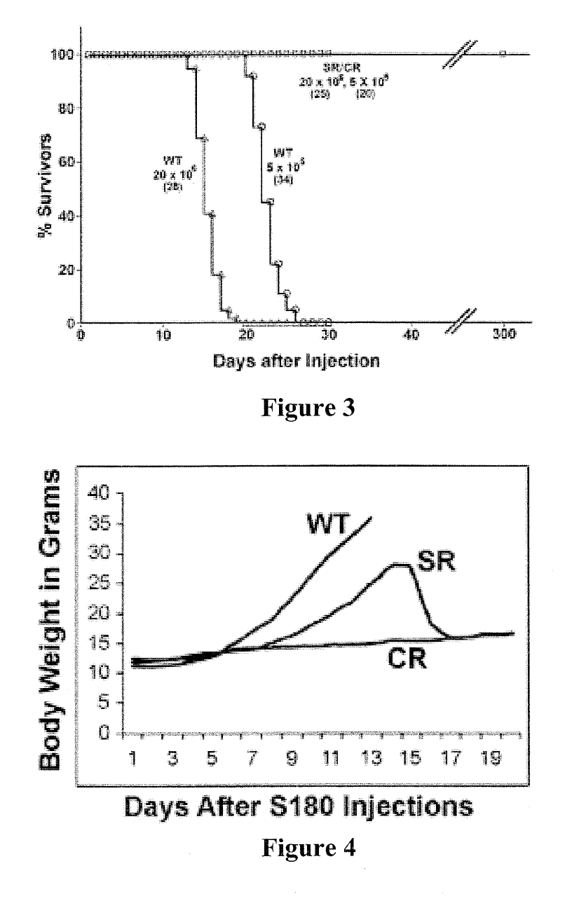

The resistance trait was transmitted independently of gender of either parent or progeny in F1 and subsequent generations. Thus, the trait is likely to be linked to one of the 19 mouse autosomes, but not to the X or Y chromosome. To determine if the resistance trait was also effective in different genetic backgrounds, the SR/CR BALB/c mice were crossed to sensitive C57BL/6 inbred mice. The trait was transmitted with a similar frequency into N1, N2 and N3 progeny in the C57BL/6 background (Table I). A similar transmission frequency was also observed in breeding the trait into a wild inbred CAST/Ei background (Table I). These results argue strongly that the trait was truly a dominant gain-of-function mutation. FIG. 3 summarizes survival data from resistant progeny compared to sensitive progeny using two different doses of S180 cells. This figure demonstrates that this trait represents a powerful phenotype of resistance to transplanted cancer cells in a dose independent manner.

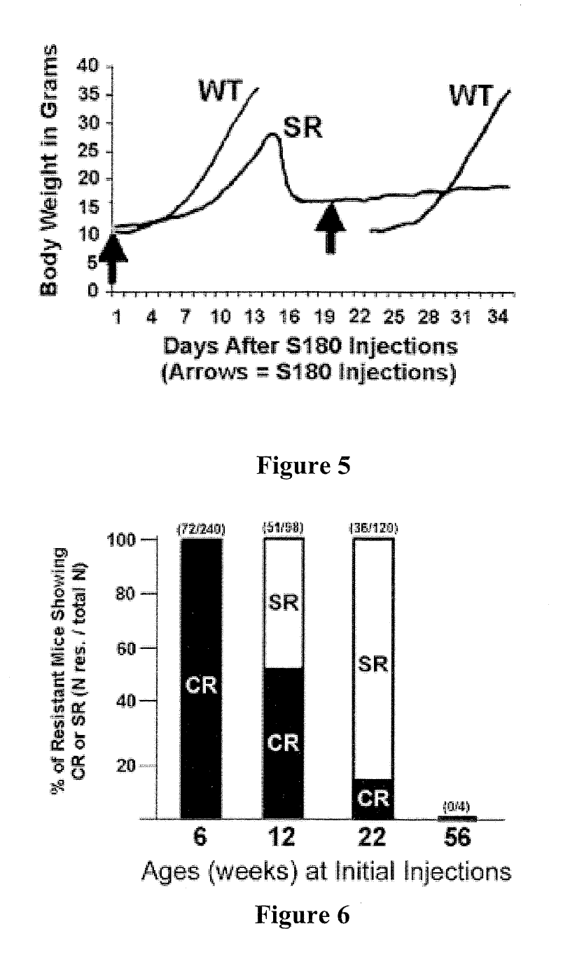

The SR/CR Phenotype Is Age-Dependent and Involves Priming. In contrast to complete resistance (CR) to S180-induced ascites, a portion of the S180-resistant mice displayed spontaneous regression (SR), dependent on the age at the first injection of S180 cells. After injection of S180 cells, SR mice developed ascites for the first 2 weeks, which rapidly disappeared in less than 24 hours (FIG. 4). The mice then became healthy and immediately resumed normal activities including mating. S180 cells in the regressed ascites were equivalent to 3 grams of solid tumor mass or 3×10⁹ of cells. The mice that underwent regression remained ascites-free, thereafter. We then determined if a repeated injection of S180 cells would induce a repeated ascites/regression in the mice that had shown ascites/regression once. However, the mice that had once undergone regression became completely protected from S180 cells, and never developed ascites again in response to subsequent i.p. injections of S180 cells (FIG. 5). The initial development of ascites suggested that the anti-cancer mechanism might not be engaged immediately in response to the implantation of cancer cells in older animals. After an initial period of latency, an anti-cancer mechanism was rapidly engaged in these mice leading to destruction of S180 cells, clearance of peritoneal lymphatic drainage and regression of ascites. The lasting protection against S180-induced ascites after initial regression suggests that the anti-cancer mechanism, after being engaged once, is primed for later engagement in response to subsequent exposures to S180 cells.

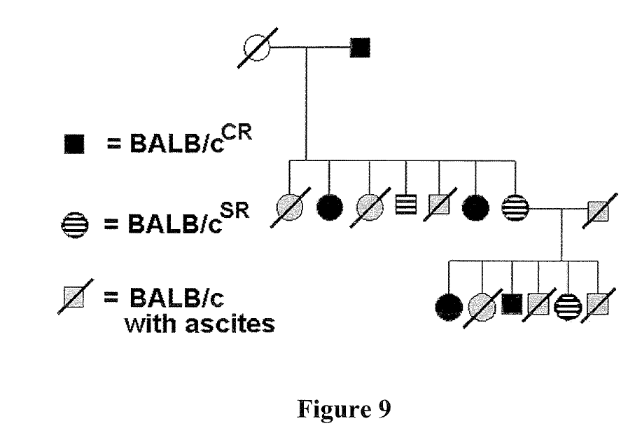

The manifestation of the CR or SR phenotypes was related to the age of mice at the time of the first injection with S180 cells. When the first injection was given at the age of 6 weeks, essentially all of the resistant mice display the CR phenotype. When the first injection of S180 cells was given at the age of 12 weeks, ˜50% of the S180 resistant mice showed the SR phenotype and the other 50% showed the CR phenotype. When the first injection of S180 cells was given at the age of 22 weeks, however, the majority of the resistant mice showed SR. In a small number of mice tested at 56 weeks, however, the first injection of S180 cells resulted in ascites and death even in mice whose offspring were cancer-resistant (FIG. 6). Genetic analysis also shows that CR and SR are derived from the same mutation locus, since the SR phenotype was inherited from CR parents, and vise versa (FIG. 9).

SR/CR Resistance Is Not Restricted to S180-Induced Ascites. To examine if the SR/CR mice would resist the formation of solid tumors from subcutaneously injected S180 cells, we injected a total of 2×10e6 S180 cells in each of two sites (one left and one right) in the shoulder regions of SR/CR mice that had been demonstrated previously to be resistant to S180-induced ascites. Four weeks after injection, visible solid tumors developed in all control mice, but not in the SR/CR mice (results not shown). Evidence for regression of solid tumor masses was also found in the SR/CR mice. Two solid tumor nodules approximately 0.3 centimeter in diameter were found on the wall of the peritoneal cavity immediately after regression of ascites in an older mouse injected i.p. with 2×10⁷ S180 cells 16 days previously. The tumors were removed, fixed and examined histologically. Significantly different from S180-derived solid tumors in the control mice, these two tumors contained isolated groups of S180 cells surrounded by extensive desmoplasia, consistent with regression of solid tumors.

To address the question of whether the SR/CR mice could resist other types of transplantable cancers, we tested the ability of the mice to eradicate transplanted cell lines and/or the ability of leukocytes from the SR/CR mice to kill different cell lines in cell culture. The results are summarized in Table II. It appears that the resistance extends to a broad array of cancer cells.

TABLE II

| | | | | | *SR/CR Cell | |
|---|---|---|---|---|---|---|
| | | | | | Killing | |
| | | | | Haplo- | in | in |
| Cell Line | Cancer Type | MHC-1 | Source | type | vivo | vitro |
| S180 | Sarcoma | − | Swiss | H2q | Yes | Yes |
| L5178Y | Lymphoma | − | DBA/2 | H2d | Yes | nd |
| MethA | Sarcoma | + | BALB/c | H2d | Yes | Yes |
| P815 | Mastocytoma | − | DBA/2 | H2d | Nd | Yes |
| LL/2 | Lung carcinoma | − | C57BL | H2b | Nd | Yes |
| BW5147.3 | T cell lymphoma | − | AKR/J | H2k | Nd | Yes |
| Hepa 1-6 | Hepatoma | − | C57BL | H2b | Nd | Yes |
| KLN 205 | Squamous cell Ca | − | DBA/2 | H2d | Nd | Yes |
| EL-4 | B cell lymphoma | + | C57BL | H2b | Nd | Yes |

*Cell killing assays (in vitro and in vivo) were performed as described in Materials and Methods
(nd = not done)

Randomly selected SR/CR mice were examined histologically for signs of autoimmune diseases. No signs of pathology were detected (results not shown), and all of the SR/CR mice showed normal behavior, normal body weight and a normal life span.

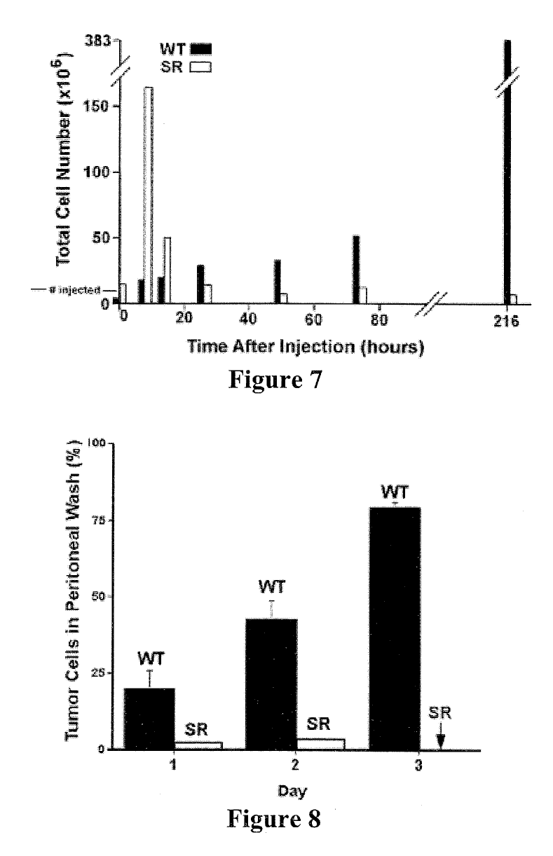

Infiltration of Host Immune Cells and Rapid Destruction of Cancer Cells via Cytolysis. To study the cell death events in the peritoneal cavity, GFP-transfected S180 cells were used for injection. At specific time points after injection of S180 cells, the peritoneal cavity of each anesthetized or sacrificed mouse was washed with either PBS or culture medium. The S180/GFP cells were readily distinguishable from leukocytes by their difference in size, morphology and by GFP fluorescence. We found that an SR/CR mouse was capable of destroying up to 20 million S180 cells in the first 12 hours. After the majority of S180 cells were destroyed, residual S180 cells could be occasionally detected in the first 48 hours, but were completely absent thereafter (FIGS. 7 and 8). The total cells in the peritoneal washes from both control and SR/CR mice were also analyzed by flow cytometry using forward scatter (cell size) on the X axis and side scatter (granularity and size) on the Y axis. At day 7, S180 cells became the dominant cell population in the peritoneal cavity of control mice, but were not detected in the SR/CR mice (data not shown). A day 4 cytoprep also showed that cancer cells were completely eliminated in the SR/CR mice (data not shown). In contrast, some leukocytes in the control mice showed apoptosis. Interestingly, 6-12 hours after injection, as many as 1.6×10e8 leukocytes migrated into the peritoneal cavity in SR/CR mice in response to the presence of S180 cells, yet disappeared after cancer cells were destroyed (FIG. 7).

In the peritoneal washes from control mice, S180 cells were scattered evenly throughout the cytoprep fields. No significant aggregation of cells was observed. In sharp contrast, S180 cells from the SR/CR mice were surrounded by immune cells forming rosettes and larger cellular aggregates (data not shown). Additionally, many S180 cells in rosettes were ruptured, suggesting a primary cytolytic event. Apoptotic morphology was not observed in the injected S180 cells.

The cells in the peritoneal washes were also examined by scanning electron microscopy (SEM). S180 cells in the peritoneal washes of control mice displayed larger diameters than leukocytes and had numerous surface microvilli (data not shown). In the peritoneal washes of the SR/CR mice challenged with 2×10e7 S180 cells for 24 hours, S180 cells displayed a variety of morphological changes including swelling, flattening and simplification of microvilli, tight contact with leukocytes, and surface erosions consistent with membrane damage (data not shown).

To verify that the destruction of cancer cells was via cell rupture, the culture peritoneal cells were recorded using time-lapse video phase contrast microscopy. In addition to formation of cell-cell aggregates, cytolytic rupture of cancer cells was also evident (see PNAS website for video clips).

T Lymphocytes Are Not Involved in the Destruction of Cancer Cells. T cells have long been believed to be the primary effector cells in host immunity against cancer. We undertook a genetic approach to determine if the resistance to S180 cells in the SR/CR mice required mature T cells. The experimental design was to determine if resistance to S180 cells occurred in an athymic nude background in which the maturation of T cells is blocked by the absence of a thymus. The phenotype of T cell absence in the nude mice is sometimes thought to be “leaky”. However, the fact that nude mice accept transplants from different species argues that this “leakiness” of T cells does not impair successful heterotransplantation. Female mice of the N3 progeny of the C57BL/6 SR/CR congeneic line were crossed to homozygous nude (foxn1⁻/foxn1⁻) males in the C57BL/6 background (S180-sensitive). All progeny from this cross carried a single copy of the recessive nude gene (foxn1⁻) and grew hair. All progeny were injected with S180 cells. Approximately 40% of these mice were ascites-free. The SR/CR females from this cross were then crossed again with homozygous nude (foxn1⁻/foxn1⁻) males. Sixteen of 31 progeny were nude mice (foxn1⁻/foxn1⁻). Upon i.p. injection of S180 cells, 10 of 16 nude mice (foxn1⁻/foxn1⁻), developed and succumbed to ascites, and 6 were ascites-free. Similar to parental nude mice, SR/CR nude mice also completely lacked thymus, consistent with impairment of T cell development (results not shown). This finding indicates that lack of T cells did not impair the SR/CR phenotype that, thus, may require other immune components.

Leukocytes of the Innate Immune System Appear to Mediate Tumor Cell Killing. By analysis of fluorescence-labeled surface markers and cell morphology in SR/CR mice, the infiltrating leukocytes found enriched in the peritoneal washes and associated with dying tumor cells were mainly leukocytes of the innate immune system, including neutrophils (PMN), macrophages and natural killer (NK) cells (results not shown). In preliminary experiments, washed peritoneal infiltrating cells were harvested from SR/CR mice and adoptively transferred into control mice prior to challenge with S180 tumor cells. Such recipient mice showed resistance to subsequently injected S180 cells, indicating that the mechanism of tumor cell killing was mediated by these infiltrating immune cells.

DISCUSSION. The SR/CR mouse model represents a unique opportunity to examine cancer/host interactions. The killing of tumor cells primarily by cytolysis in SR/CR mice was extremely rapid and effective, yet was achieved with profound selectivity, with most normal cells being unharmed. The efficiency of this cell killing has a number of striking features. Once primed by the initial challenge of S180 cells, the SR/CR mice could withstand repeated daily challenge of more than 2×10⁷ S180 cells, and could also remain ascites-free after a single challenge of up to 3×10⁹ S180 cells (10% of body weight). Tumor cell killing was accompanied by a dramatic migration of leukocytes that form rosettes and aggregates with cancer cells. Following cell contact, tumor cells undergo lysis. This cellular debris was then engulfed by peritoneal macrophages. The mice were then subsequently tumor-free. The resistance mechanism appears to involve cells of the innate immune system and is not dependent on T cell function. Histological examination of tissues in SR/CR mice showed normal morphology. Although life-span studies have not been completed, there was no sign of a shortened life span in SR/CR mice.

Several intriguing implications derive from the properties of the SR/CR mouse. First, this model demonstrates the existence of a host resistance gene that can prevent the growth of advanced, MHC-negative cancers. The existence of host cancer-resistance genes has been postulated to be one explanation for the existence of individuals in the human population who fail to develop cancers, in spite of prolonged and intense exposure to carcinogens (Balmain, A. & Nagase, H. (1998). Trends Genet. 14, 139-144). The gene(s) responsible for the SR/CR phenotype may well be an example of such a resistance gene that might have a direct human ortholog. Second, the concept of immune surveillance has been debated for decades and has been difficult to prove, although recent studies have lent support to this concept (Dunn, G. P. et al. (2002) Nat. Immunol. 3, 991-998). The SR/CR mouse may also provide a potential example of such a surveillance mechanism. Third, the alteration in the type of response seen with age in these mice suggests an intriguing possibility. The appearance of cancer in older individuals at a much higher frequency may not solely be due to the accumulation of mutations in individual pre-neoplastic cells. This mouse model suggests that there may also be host resistance mechanisms that decline with age. Fourth, the rare phenomenon of spontaneous regression of cancers has been documented in humans, but has been difficult to study due to lack of an appropriate animal model. The SR/CR mouse may provide such a model and allow identification of the cellular and genetic machinery necessary to reject a fully developed malignancy.

Mouse models of immune-mediated rejection of transplanted tumors through T cell-mediated recognition or through abrogation of immune suppressive cytokines have been clearly demonstrated (e.g., Gorelik, L. & Flavell, R. A. (2001) Nature Med. 7, 1118-1122). The SR/CR mouse, however, provides an example of a unique genetically-determined mechanism of resistance independent of T cells. Further studies of the underlying genetic, cellular and biochemical mechanisms in the SR/CR mouse should yield a deeper understanding of how tumor cells evade host immune rejection. Further, the ability of adoptively transferred infiltrating leukocytes from SR/CR mice to protect control mice from S180 cells (seen in preliminary studies) may suggest a potentially feasible strategy for treatment of advanced cancers that could be translatable into human patients.

### EXAMPLE 2

Pedigree analysis of the CR and SR phenotypes. Outcross progeny were first injected with 2×10⁶ S180 cells at 12 weeks of age. The pedigree (Shown in FIG. 9) indicates that the SR trait was inherited from a CR parent and the CR trait was inherited from an SR parent. The ratio of SR/CR was age-dependent determined by the time of the initial injection of cancer cells. Males are represented by squares and females by circles. Dark squares and circles are CR mice. Hatched squares and circles are SR mice. Clear circles represent control mice. Slashed squares and circles are S180-sensitive progeny.

### EXAMPLE 3

Histologic appearance of S180 tumor cells in intraperitoneal implants in sensitive and resistant mice. S180 cells (20×10⁶) injected i.p. 16 days before necropsy generated widespread ascites and peritoneal implants in wild type BALB/c mice, as typified by the metastatic implant near the renal capsule in (A). On the other hand, while a similarly injected SR/CR mouse failed to develop ascites cancers, a few small nodules attached to the peritoneal surface were noted and dissected for histology. These nodules were composed of small numbers of swollen cancer cells surrounded by a dense fibroblastic proliferation (desmoplasia) (data not shown).

The foregoing is illustrative of the present invention, and is not to be construed as limiting thereof. The invention is defined by the following claims, with equivalents thereof to be included therein.

That which is claimed is:

1. A mouse that exhibits the phenotype of resistance to the development of ascites when 2×10⁶ S180 tumor cells are injected into the peritoneal cavity of said mouse at 6 weeks of age.

2. The mouse according to claim 1, wherein said mouse is male.

3. The mouse according to claim 1, wherein said mouse is female.

4. The mouse according to claim 1, wherein said mouse exhibits the phenotype of complete resistance to the development of ascites.

5. The mouse according to claim 1, wherein said mouse exhibits the phenotype of spontaneous regression of ascites.

6. The mouse according to claim 1, wherein said mouse is a BALB/c mouse.

7. A mouse colony comprising a plurality of mice of claim 1.

8. An isolated cell isolated from a mouse of claim 1.

9. The cell of claim 8, wherein said cell is selected from the group consisting of blood cells, hepatic cells, pancreatic cells, muscle cells, neural cells, skin cells, bone cells, hematopoietic stem cells, embryonic stem cells, egg cells and sperm cells.

10. A cell culture consisting essentially of isolated cells of claim 8

11. A tissue culture derived from an isolated cell of claim 8.

12. A method of producing a cancer-resistant mouse, comprising the steps of:
(a) providing a first parent mouse and a second parent mouse, wherein said first parent mouse exhibits the phenotype of resistance to the development of ascites when 2×10⁶ S180 tumor cells are injected into the peritoneal cavity of said mouse at 6 weeks of age; and then
(b) crossing said first and second parent mice with one another to produce a progeny mouse that exhibits a phenotype of resistance to the development of ascites when 2×10⁶ S180 tumor cells are injected into the peritoneal cavity of said mouse at 6 weeks of age.

13. The method of claim 12, wherein said first parent mouse is a BALB/c mouse.

14. The method of claim 12, wherein said second parent mouse is a BALB/c mouse.

15. The method of claim 12, wherein said first parent mouse is male and said second parent mouse is female.

16. The method of claim 12, wherein said first parent mouse is female and said second parent mouse is male.

17. The method of claim 12, wherein said second parent mouse is a wild-type mouse.

18. The method of claim 12, wherein said second parent mouse exhibits the phenotype of resistance to the development of ascites when 2×10⁶ S180 tumor cells are injected into the peritoneal cavity of said mouse at 6 weeks of age.

19. A method of screening a compound for carcinogenic activity, comprising:
(a) providing an mouse according to claim 1;
(b) injecting cancer cells into said mouse;
(c) administering said compound to said animal; and
(d) determining whether said cancer cells grow in said animal by an amount greater than that seen in a corresponding control mouse that has been administered the same cancer cells but not been administered said compound, greater growth of said cancer cells indicating said compound may be carcinogenic.

20. A method of screening a compound for anti-carcinogenic activity, comprising:
(a) providing an mouse according to claim 1;
(b) injecting cancer cells into said mouse;
(c) administering said compound to said animal; and
(d) determining whether said cancer cells grow in said animal by an amount less than that seen in a corresponding control mouse that has been administered the same cancer cells but not been administered said compound, less growth of said cancer cells indicating said compound may be anti-carcinogenic.

21. A method of screening a compound for immune suppressing activity, comprising:
(a) providing an mouse according to claim 1;
(b) injecting cancer cells into said mouse;
(c) administering said compound to said animal; and
(d) determining whether said cancer cells grow in said animal by an amount greater than that seen in a corresponding control mouse that has been administered the same cancer cells but not been administered said compound, greater growth of said cancer cells indicating said compound may have immune suppressing activity

22. A method of screening a compound for immune stimulating activity, comprising:
(a) providing an mouse according to claim 1;
(b) injecting cancer cells into said mouse;
(c) administering said compound to said animal; and
(d) determining whether said cancer cells grow in said animal by an amount less than that seen in a corresponding control mouse that has been administered the same cancer cells but not been administered said compound, less growth of said cancer cells indicating said compound may have immune stimulating activity.
